(19) United States
(12) Patent Application Publication
(10) Pub. No.: US 2025/0295615 A1
(43) Pub. Date: Sep. 25, 2025

(54) COMBINATIONS OF CURCUMIN AND URSOLIC ACID AND USES THEREOF

(71) Applicant: BOARD OF REGENTS, THE UNIVERSITY OF TEXAS SYSTEM, Austin, TX (US)

(72) Inventors: Michael A. LISS, Austin, TX (US); John DIGIOVANNI, Austin, TX (US); Achinto SAHA, Austin, TX (US)

(21) Appl. No.: 18/711,040

(22) PCT Filed: Nov. 18, 2022
(86) PCT No.: PCT/US2022/050401

§ 371 Date: May 16, 2024

Related U.S. Application Data
(60) Provisional application No. 63/280,831, filed on Nov. 18, 2021.

(51) Int. Cl. A61K 31/19; A61K 31/12

(52) U.S. Cl. CPC A61K 31/19; A61K 31/12

(57) ABSTRACT

The disclosed subject matter relates to compostions comprising ursolic acid or pharmaceutically acceptable salts thereof and curcumin or pharmaceutically acceptable salts thereof. Further, disclosed herein are methods of using ursolic acid or pharmaceutically acceptable salts thereof and curcumin or pharmaceutically acceptable salts thereof for treating, inhibiting initiation, inhibiting progression, and/or inhibiting metastasis of cancer, such as prostate cancer, in a subject.

### CROSS REFERENCE TO RELATED APPLICATIONS

This application is a U.S. National Stage application filed under 35 U.S.C. § 371 of PCT/US2022/050401, filed Nov. 18, 2022, which claims the benefit of priority to U.S. Provisional Application No. 63/280,831 filed Nov. 18, 2021, each of which is incorporated by reference herein in their entirety.

This invention was made with government support under Grant no. R01 CA228404 and R01 CA164159 awarded by the National Institutes of Health. The government has certain rights in the invention.

### BACKGROUND

There are an estimated 1.5 million men living with prostate cancer (PCa). The average age for diagnosis of PCa is 66; however, the onset of preclinical disease may occur in adults as early as 30 years of age. Guidelines currently emphasize closely monitoring low-grade prostate cancer, which is the most common diagnosis. Since there can be a considerable time for the disease to progress to clinically evident cancer, there is ample opportunity for chemopreventive strategies to be applied for the successful management of PCa. Finasteride (a 5α-reductase inhibitor) has been evaluated as a potential chemopreventive agent for PCa in the Prostate Cancer Prevention Trial (PCPT). A 24.8% reduction in prostate tumor prevalence was observed over a 7-year period in men taking this drug daily, although the finasteride administered group showed higher rates of sexual side effects and higher grades of prostate tumors. However, there are no drugs currently used to slow the progression of low-grade PCa.

Interest in the use of phytochemicals for the prevention or treatment of various cancers, including PCa has grown considerably in recent years. A number of agents, including curcumin (CURC), ursolic acid (UA), green tea (or EGCG), metformin, resveratrol (RES), various NSAIDS and 6-shogaol (6-SHO) have shown potential chemopreventive effects in either animal models of PCa or in clinical studies in men. Many of these agents target inflammatory signaling pathways including STAT3 and NFkB in addition to other cell signaling pathways associated with PCa development and progression. Moreover, as with standard of care therapies, the administration of phytochemical combinations offers considerable promise to improve outcomes. In fact, when compared to treatment with a single agent, combination therapies provide several advantages including better efficacy due to targeting/modulation of multiple cell signaling pathways, lower toxicity due to lower required doses and potentially reducing the development of resistance to therapy.

Several studies have evaluated the efficacy of phytochemicals and their combinations as a preventive or therapeutic measure in PCa as well as other tumor types. For example, the combination of CURC and phenethyl isothiocyanate strongly inhibited PC-3 xenograft tumor growth compared to the individual agents. In a randomized double blind controlled study in men who received prostate biopsies, but were not found to have prostate cancer, the combination of CURC with soy isoflavones significantly suppressed PSA production. Combinations have also shown promise of increased activity in other cancer sites. For example, a combination of CURC with phospho-sulindac showed better inhibitory activity in a xenograft model using A549 cells than either agent alone. In another study, the combination of RES and CURC showed a stronger inhibitory effect on growth of head and neck cancer cells both in vitro and in vivo. Phytochemicals such as RES, UA, CURC and 6-SHO are established anti-inflammatory agents and have been shown to inhibit the growth of many cancers, including breast, prostate, colon and liver both in cell culture and in preclinical animal models. These compounds have also shown inhibitory activity against both STAT3 and NFκB signaling as part of their anticancer mechanism of action. In addition, these compounds are also reported to activate AMPK signaling pathways. Recent evidence also suggests an effect of these phytochemicals on the CXCL12/CXCR4 signaling axis which plays a significant role in the progression of PCa. CURC, UA, RES and 6-SHO also have effects on mitochondrial function. Thus, there is ample evidence both in PCa as well as other cancer types that combinations of phytochemicals can lead to enhanced efficacy for inhibition of tumor growth.

Even though over half of all cancer patients report taking dietary supplements after they were told they have cancer, there is a lack of clinical evidence that dietary supplements can reduce cancer progression. Thus, effective evidence-based treatments with limited side effects are urgently needed. The compositions and methods disclosed herein address these and other needs.

### SUMMARY

In accordance with the purposes of the disclosed materials and methods, as embodied and broadly described herein, the disclosed subject matter, in one aspect, relates to compounds, compositions and methods of making and using compounds and compositions. In specific aspects, the disclosed subject matter relates to compostions comprising ursolic acid or pharmaceutically acceptable salts thereof and curcumin or pharmaceutically acceptable salts thereof. Further, disclosed herein are methods of using ursolic acid or pharmaceutically acceptable salts thereof and curcumin or pharmaceutically acceptable salts thereof for treating, inhibiting initiation, inhibiting progression, and/or inhibiting metastasis of cancer, such as prostate cancer, in a subject.

Additional advantages will be set forth in part in the following description and in part will be obvious from the description or may be learned by practicing the aspects described below. The advantages described below will be realized and attained by the elements and combinations pointed out in the appended claims. It is to be understood that the foregoing general description and the following detailed description are exemplary and explanatory only and are not restrictive.

### BRIEF DESCRIPTION OF FIGURES

The accompanying figures, which are incorporated in and constitute a part of this specification, illustrate several aspects described below.

FIG. 1 is a consort diagram. Displayed is a phase 1 design where each study group that include curcumin, ursolic acid, and the combination are stand-alone 3 by 3 study designs. If any side-effects were identified, another 3 subjects were enrolled. The side effects for each study group are listed below the group name and sample size (n=3). The arrow pointing left to right indicated we enrolled a second group of 3 patients. If less than 3 subjects had side effects at the particular dose, then that dose is considered safe to proceed. In the case of natural products, very high doses such as several grams per day have been reported so the minimum tolerated dose (MTD) studies did not apply to this trial.

FIG. 2 displays bar charts of the plasma levels of ursolic acid, curcumin, and curcumin metabolites (urcumin sulfate and curcumin glucuronide). The first 6 (1-6) subjects took ursolic acid (UA) 300 mg/day with modest absorption of ursolic acid and only one subject with significant levels over 40 mg. The next 6 subjects (subjects 7-12) took 1200 mg/day of curcumin. Minimal amounts of the parent curcumin compound and large amounts of curcumin glucuronide indicating curcumin was in its glucuronidated form from metabolism were noted. The last 6 subjects (13-18) were in the combination group (CurcUA) which noted a statistically significant increase in ursolic acid and curcumin glucuronide.

FIG. 3 displays the changes in the gut microbiome over a 2-week period. At the top of the graph, the study group (curcumin, ursolic acid, or combination) is displayed along with the variable regain (V1-2 or V3-4) that was sequenced. The variable regions can provide difference results therefore we display all the data for visual comparison, in that, if consistent the result is more robust. The figure legend on the right shows the change (delta) in the value over the two weeks after normalization. The circles correspond to specific p-values. The top box shows specific taxa of bacteria that change over two weeks in each group. The bottom box shows the metabolic pathways that change over time.

FIG. 4 is a schematic of the study design.

FIG. 5 contains extracted ion chromatogram of Standard and IS.

FIG. 6 contains MS/MS spectra of Standards/IS.

FIG. 7 is a calibration plot of Standards.

FIG. 8 displays four separate box plots noting alpha diversity, which measures the diversity of species. The top two box plots represent the Simpson's reciprocal index which quantifies biodiversity by considering richness and evenness of the microbiome representing overall biodiversity. Richness represents the number of species and evenness represents the proportional abundance of those species. We use two sets of variable regions (V1-V2 and V3-V4) to insure robustness in the analysis. The Shannon index is a commonly used and considered a standard diversity measure by dividing the number species in a group by the total number of individuals in the community. The box plots represent these values as pairs (visit.2=baseline represented by circles and visit.4=end of study, represented by diamonds). The circles (baseline) are connected to the end of the 2 weeks study levels (diamonds) by a line. The box represents the 95% confidence interval and line represents the median. In general, curcumin and ursolic acid may lower overall alpha diversity, whereas the combination treatment improves overall diversity. Using the paired t-test, the difference of pre and post intervention were compared. All V1-V2 values (Shannon or Simpson reciprocal) showed not statistical differences (all p>0.05). Statistical differences in V3-V4 curcumin reduction in alpha diversity (p=0.004) and a trend for improvement of alpha diversity for the combination (CurcUA, p=0.06) were not differentiated.

FIG. 9 shows microbiome beta diversity. The top panels represent the principal coordinate analysis (PCoA) using Bray-Curtis calculated distances that represent similarity between groups (left V1-V2 and right V3-V4). The change before and after treatment of curcumin (yellow), ursolic acid (blue), or the combination (green) were compared. The circle represents the baseline composition, and the diamond represents the gut microbiome composition after treatment. To the right of each PCoA plot is summarized the direction and distance from a fixed point to compare the differences in subjects. The direction does not represent a good or bad result, only different composition. Curcumin may be driving the beta diversity because in both curcumin and the combination (CurcUA) seem to have a directional component to changing diversity though they seem to be in opposite directions.

### DETAILED DESCRIPTION

The materials, compounds, compositions, and methods described herein may be understood more readily by reference to the following detailed description of specific aspects of the disclosed subject matter and the Examples and Figures included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### Definitions

In this specification and in the claims that follow, reference will be made to a number of terms, which shall be defined to have the following meanings:

As used herein the singular forms “a,” “an,” and “the” include plural referents unless the context clearly dictates otherwise. Thus, for example, reference to “a composition” includes mixtures of two or more such compositions, reference to “an inhibitor” includes mixtures of two or more such inhibitors, and the like.

As used herein “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

“Optional” or “optionally” means that the subsequently described event or circumstance can or cannot occur, and that the description includes instances where the event or circumstance occurs and instances where it does not.

The term “administration” and variants thereof in reference to a composition means introducing the composition into the system of the subject in need of treatment. When a composition disclosed herein is provided in combination with one or more other active agents (e.g., a cytotoxic agent, etc.), “administration” and its variants are each understood to include concurrent and sequential introduction of the composition thereof and other agents.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means decreasing the amount of tumor cells relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As used herein, “treatment” refers to obtaining beneficial or desired clinical results. Beneficial or desired clinical results include, but are not limited to, any one or more of: alleviation of one or more symptoms (such as tumor growth), diminishment of extent of cancer, stabilized (i.e., not worsening) state of cancer, delaying spread (e.g., metastasis) of the cancer, delaying occurrence or recurrence of cancer, delay or slowing of cancer progression, amelioration of the cancer state, and remission (whether partial or total).

The term “subject” preferably refers to a human in need of treatment with an anticancer agent or treatment for any purpose, and more preferably a human in need of such a treatment to treat cancer, or a precancerous condition or lesion. However, the term “subject” can also refer to non-human animals, preferably mammals such as dogs, cats, horses, cows, pigs, sheep and non-human primates, among others, that are in need of treatment with an anticancer agent or treatment.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

As used herein, the term “composition” is intended to encompass a product comprising the specified ingredients in the specified amounts, as well as any product which results, directly or indirectly, from combination of the specified ingredients in the specified amounts.

References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight component Y, X and Y are present at a weight ratio of 2:5, and are present in such ratio regardless of whether additional components are contained in the mixture.

A weight percent (wt. %) of a component, unless specifically stated to the contrary, is based on the total weight of the formulation or composition in which the component is included.

The compositions used herein can be substantially pure. As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

A “pharmaceutically acceptable” component is one that is suitable for use with humans and/or animals without undue adverse side effects (such as toxicity, irritation, and allergic response) commensurate with a reasonable benefit/risk ratio.

“Pharmaceutically acceptable salt” refers to a salt that is pharmaceutically acceptable and has the desired pharmacological properties. Such salts include those that may be formed where acidic protons present in the compounds are capable of reacting with inorganic or organic bases. Suitable inorganic salts include those formed with the alkali metals, e.g., sodium, potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). When two acidic groups are present, a pharmaceutically acceptable salt may be a mono-acid-mono-salt or a di-salt; similarly, where there are more than two acidic groups present, some or all of such groups can be converted into salts.

“Pharmaceutically acceptable excipient” refers to an excipient that is conventionally useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

A “pharmaceutically acceptable carrier” is a carrier, such as a solvent, suspending agent or vehicle, for delivering the disclosed compounds to the patient. The carrier can be liquid or solid and is selected with the planned manner of administration in mind. Liposomes are also a pharmaceutical carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. In reference to cancers or other unwanted cell proliferation, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay occurrence and/or recurrence. An effective amount can be administered in one or more doses. In the case of cancer, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

Reference will now be made in detail to specific aspects of the disclosed materials, compounds, compositions, articles, and methods, examples of which are illustrated in the accompanying Examples and Figures.

### Methods

A combination of UA+CURC was identified from a high through-put screen of a natural product library to have synergistic ATP depletion in PCa cells as well as synergistic inhibition of PCa tumor growth in vivo. Still, major challenges for translation of results in preclinical models to human trials using natural products and dietary supplements include good manufacturing practices (GMP), known active ingredients, bioavailability, and clinical trial rigor. These challenges are addressed herein using a combination of UA+CURC in an academically run, Phase I clinical trial with known active ingredients and enhanced bioavailability using GMP protocols.

Thus, the disclosed subject matter, in one aspect, relates to a method of treating, inhibiting initiation, inhibiting progression, and/or inhibiting metastasis of cancer in a subject, by administrating ursolic acid or a pharmaceutically acceptable salt thereof and curcumin or a pharmaceutically acceptable salt thereof.

The disclosed compositions can be administered either sequentially or simultaneously in separate or combined pharmaceutical or nonpharmaceutical formulations. When one or more of the disclosed compositions is used in combination with a second therapeutic agent, the dose of the composition can be either the same as or differ from that when the composition is used alone. Appropriate doses will be readily appreciated by those skilled in the art.

Administration can be accomplished by any suitable method and technique presently or prospectively known to those skilled in the art. For example, the ursolic acid and curcumin can be used as is or formulated in a physiologically or pharmaceutically acceptable form and administered by any suitable route known in the art including, for example, oral routes of administration, topical or skin applications, mouth gargling, chewing gum, and nasal spray. Administration of the disclosed compositions can be a single administration, or at continuous or distinct intervals as can be readily determined by a person skilled in the art.

The compositions disclosed herein can also be administered utilizing slow-release capsules, implantable pumps, and biodegradable containers. These delivery methods can, advantageously, provide a uniform dosage over an extended period of time.

The compositions disclosed herein can be formulated according to known methods for preparing pharmaceutical or nonpharmaceutical (nutritional/supplement) compositions. Formulations are described in detail in a number of sources which are well known and readily available to those skilled in the art. For example, Remington's Pharmaceutical Science by E.W. Martin (1995) describes formulations that can be used in connection with the disclosed methods. In general, the compositions disclosed herein can be formulated such that an effective amount of each component in the composition is combined with a suitable carrier in order to facilitate effective administration of the composition. The compositions used can also be in a variety of forms. These include, for example, solid, semi-solid, and liquid dosage forms, such as tablets, pills, powders, liquid solutions or suspension, suppositories, injectable and infusible solutions, and sprays. The form depends on the intended mode of administration and therapeutic application. Examples of carriers or diluents for use with the compositions include ethanol, dimethyl sulfoxide, glycerol, alumina, starch, saline, and equivalent carriers and diluents. To provide for the administration of such dosages for the desired therapeutic treatment, compositions disclosed herein can advantageously comprise from 0.1% and 100% by weight of the total of one or more of the subject compositions based on the weight of the total composition including carrier or diluent.

The disclosed compositions, or the various active components thereof, can be administered with tablets, troches, pills, capsules, and the like. Such formulations can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the composition, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the composition can be incorporated into sustained-release preparations and devices.

Solutions of the active agent can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of compositions.

Alternatively, the composition can be suspended or emulsified in a non-solvent to form a suspension or emulsion. Other ingredients or components such as stabilizing agents, dyes, and agents assisting with the drying process may optionally be added at this stage. Examples of liquid preparations include, but are not limited to, aqueous, organic, or aqueous-organic solutions, suspensions, and emulsions.

The dosage ranges for the administration of the disclosed compositions are those large enough to produce the desired effect in which the symptoms or disorder are affected. The dosage should not be so large as to cause adverse side effects. Generally, the dosage will vary with the age, condition, sex, and extent of the disease in the subject. The dosage can be adjusted by the individual physician in the event of any counterindications. Dosage can vary, and can be administered in one or more dose administrations daily, for one or several days.

The dose administered to a subject, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

### Cancers

The compositions disclosed herein can be used to treat or inhibit prostate cancer. Other examples of cancers that can be treated according to the methods disclosed herein are adrenocortical carcinoma, adrenocortical carcinoma, cerebellar astrocytoma, basal cell carcinoma, bile duct cancer, bladder cancer, bone cancer, brain tumor, breast cancer, Burkitt's lymphoma, carcinoid tumor, central nervous system lymphoma, cervical cancer, chronic myeloproliferative disorders, colon cancer, cutaneous T-cell lymphoma, endometrial cancer, ependymoma, esophageal cancer, gallbladder cancer, gastric (stomach) cancer, gastrointestinal carcinoid tumor, germ cell tumor, glioma, hairy cell leukemia, head and neck cancer, hepatocellular (liver) cancer, hypopharyngeal cancer, hypothalamic and visual pathway glioma, intraocular melanoma, retinoblastoma, islet cell carcinoma (endocrine pancreas), laryngeal cancer, lip and oral cavity cancer, liver cancer, medulloblastoma, Merkel cell carcinoma, squamous neck cancer with occult mycosis fungoides, myelodysplastic syndromes, myelogenous leukemia, nasal cavity and paranasal sinus cancer, nasopharyngeal cancer, neuroblastoma, non-small cell lung cancer, oral cancer, oropharyngeal cancer, osteosarcoma, ovarian cancer, pancreatic cancer, paranasal sinus and nasal cavity cancer, parathyroid cancer, penile cancer, pheochromocytoma, pineoblastoma and supratentorial primitive neuroectodermal tumor, pituitary tumor, plasma cell neoplasm/multiple myeloma, pleuropulmonary blastoma, prostate cancer, rectal cancer, renal cell (kidney) cancer, retinoblastoma, rhabdomyosarcoma, salivary gland cancer, Ewing's sarcoma, soft tissue sarcoma, Sezary syndrome, skin cancer, small cell lung cancer, small intestine cancer, supratentorial primitive neuroectodermal tumors, testicular cancer, thymic carcinoma, thymoma, thyroid cancer, transitional cell cancer of the renal pelvis and ureter, trophoblastic tumor, urethral cancer, uterine cancer, vaginal cancer, vulvar cancer, Waldenström's macroglobulinemia, and Wilms' tumor.

In some additional examples, the cancer is selected from prostate cancer, breast cancer, brain cancer, cervical cancer, chronic myeloproliferative disorder, colorectal cancer, Ewing's sarcoma, gastrointestinal cancer, glioma, leukemia, lung cancer, lymphoma, endometrial cancer, melanoma, multiple myeloma, myelodysplastic syndrome, myeloproliferative neoplasm, pancreatic cancer, plasma cell neoplasm (myeloma), prostate cancer, ovarian cancer, osteosarcoma, skin cancer, testicular cancer, and thyroid cancer.

### Compostions

Also disclosed herein, in another aspect, is a composition comprising ursolic acid or a pharmaceutically acceptable salt thereof and curcumin or a pharmaceutically acceptable salt thereof.

The disclosed compositions can also include a pharmaceutically acceptable carrier and/or excipient. Pharmaceutically acceptable carriers can include, but are not limited to, inert diluents, assimilable edible carriers, binders, excipients, disintegrating agents, sweetening agents, lubricants, or flavoring agents. Examples of suitable aqueous and nonaqueous carriers, diluents, inert diluents, solvents, assimilable edible carriers, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

In specific examples, the pharmaceutically acceptable carrier can include a binder, excipient, disintegrating agent, sweetening agent, lubricant, flavoring agent, inert diluent, assimilable edible carrier, or any combination thereof.

In some examples, binder can include gum tragacanth, acacia, corn starch, gelatin, or any combination thereof. In further embodiments, excipients can include dicalcium phosphate, lactose, starch, cellulose, milk sugar, or high molecular weight polyethylene glycols. In certain embodiments, disintegrating agent can include corn starch, potato starch, alginic acid, or any combination thereof. In specific embodiments, sweetening agent can include sucrose, fructose, lactose, aspartame, or any combination thereof. In some embodiments, lubricant can include magnesium stearate. In further embodiments, flavoring agent can include peppermint, oil of wintergreen, cherry flavoring, or any combination thereof. In certain embodiments, inert diluent can include anhydrous lactose, lactose monohydrate, sugar alcohols, such as sorbitol, xylitol, or mannitol, or any combination thereof. In specific embodiments, assimilable edible carrier can include polysaccharides, polymers, pectin, polypeptides, or any combination thereof.

The ursolic acid or a pharmaceutically acceptable salt thereof and curcumin or a pharmaceutically acceptable salt thereof can be used in therapeutically effective amounts. Effective amounts of ursolic acid or a pharmaceutically acceptable salt thereof and curcumin or a pharmaceutically acceptable salt thereof for treating a mammalian subject can include about 0.001 to about 10,000 mg/Kg of body weight of the subject/day, such as from about 0.01 to about 1000 mg/Kg/day, or from about 10 to about 100 mg/Kg/day. The doses can be acute or chronic. A broad range of disclosed composition dosages are believed to be both safe and effective.

### Dose

In some examples, the therapeutically effective amount of ursolic acid or a pharmaceutically acceptable salt thereof can be from 1 to 500, e.g., from 1 to 200, 200 to 400, 400 to 600, 600 to 800, or 800 to 1000 mg in a pill. In certain examples, the therapeutically effective amount of the ursolic acid or a pharmaceutically acceptable salt thereof can be from 1 to 100, 100 to 200, 200 to 300, 300 to 400, 400 to 500, 500 to 600, 600 to 700, 700 to 800, 800 to 900, or 900 to 1000 mg in a pill. Further, the therapeutically effective amount of ursolic acid or a pharmaceutically acceptable salt thereof can be from 1 to 25, 25 to 75, 75 to 125, 125 to 175, 175 to 225, 225 to 275, 275 to 325, 325 to 375, 375 to 425, 425 to 475, 475 to 525, 525 to 575, 575 to 625, 625 to 675, 675 to 725, 725 to 775, 775 to 825, 825 to 875, 875 to 925, 925 to 975, or 975 to 1,000 mg in a pill.

In some examples, the therapeutically effective amount of the ursolic acid or a pharmaceutically acceptable salt thereof can be from 1 to 1,000, 1,000 to 2,000, 2,000 to 3,000, 3,000 to 4,000, or 4,000 to 5,000 mg per day. In certain examples, the therapeutically effective amount of the disclosed compositions can be from 1 to 500, 500 to 1,000, 1,000 to 1,500, 1,500 to 2,000, 2,000 to 2,500, 2,500 to 3,000, 3,000 to 3,500, 3,500 to 4,000, 4,000 to 4,500, or 4,500 to 5,000 mg per day. Further, the therapeutically effective amount of the ursolic acid or a pharmaceutically acceptable salt thereof can be from 1 to 200, 200 to 400, 400 to 600, 600 to 800, 800 to 1,000, 1,000 to 1,200, 1,200 to 1,400, 1,400 to 1,600, 1,600 to 1,800, 1,800 to 2,000, 2,000 to 2,200, 2,200 to 2,400, 2,400 to 2,600, 2,600 to 2,800, 2,800 to 3,000, 3,000 to 3,200, 3,200 to 3,400, 3,400 to 3,600, 3,600 to 3,800, 3,800 to 4,000, 4,000 to 4,200, 4,200 to 4,400, 4,400 to 4,600, 4,600 to 4,800, or 4,800 to 5,000 mg per day.

In some examples, the therapeutically effective amount of the ursolic acid or a pharmaceutically acceptable salt thereof can be from 1 to 200, 200 to 400, 400 to 600, 600 to 800, or 800 to 1,000 mg/kg. In certain examples, the therapeutically effective amount of the ursolic acid or a pharmaceutically acceptable salt thereof can be from 1 to 100, 100 to 200, 200 to 300, 300 to 400, 400 to 500, 500 to 600, 600 to 700, 700 to 800, 800 to 900, or 900 to 1,000 mg/kg. Further, the therapeutically effective amount of the ursolic acid or a pharmaceutically acceptable salt thereof can be from 1 to 25, 25 to 75, 75 to 125, 125 to 175, 175 to 225, 225 to 275, 275 to 325, 325 to 375, 375 to 425, 425 to 475, 475 to 525, 525 to 575, 575 to 625, 625 to 675, 675 to 725, 725 to 775, 775 to 825, 825 to 875, 875 to 925, 925 to 975, or 975 to 1,000 mg/kg.

In some examples, the therapeutically effective amount of the ursolic acid or a pharmaceutically acceptable salt thereof can be from 1 to 50, 50 to 100, 100 to 150, or 150 to 200 mg/kg per day. In certain examples, the therapeutically effective amount of the ursolic acid or a pharmaceutically acceptable salt thereof can be from 1 to 25, 25 to 50, 50 to 75, 75 to 100, 100 to 125, 125 to 150, 150 to 175, or 175 to 200 mg/kg per day. Further, the therapeutically effective amount can be from 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, or 190 to 200 mg/kg per day.

In specific aspects the amount of ursolic acid or pharmaceutically acceptable salts thereof can be 300 mg per day.

In some examples, the therapeutically effective amount of curcumin or a pharmaceutically acceptable salt thereof can be from 1 to 10,000, e.g., from 1 to 1000, 1000 to 5000, 5000 to 10000, 1 to 5000, or 100 to 8000 mg in a pill. In certain examples, the therapeutically effective amount of the curcumin or a pharmaceutically acceptable salt thereof can be from 100 to 1000, 1000 to 2000, 2000 to 3000, 3000 to 4000, 4000 to 5000, 5000 to 6000, 6000 to 7000, 7000 to 8000, 8000 to 9000, or 9000 to 10000 mg in a pill. Further, the therapeutically effective amount of curcumin or a pharmaceutically acceptable salt thereof can be from 1 to 10000, 200 to 8000, 500 to 6000, 1000 to 4000, 2000 to 3000, or about 1200 mg in a pill.

In some examples, the therapeutically effective amount of the curcumin or a pharmaceutically acceptable salt thereof can be from 1 to 1,000, 1,000 to 2,000, 2,000 to 3,000, 3,000 to 4,000, 4,000 to 5,000, 5000 to 6000, 6000 to 7000, or 8000 mg per day. In certain examples, the therapeutically effective amount of the curcumin or pharmaceutically acceptable salt thereof can be from 1 to 500, 500 to 1,000, 1,000 to 1,500, 1,500 to 2,000, 2,000 to 2,500, 2,500 to 3,000, 3,000 to 3,500, 3,500 to 4,000, 4,000 to 4,500, or 4,500 to 5,000 mg per day. Further, the therapeutically effective amount of the curcumin or a pharmaceutically acceptable salt thereof can be from 1 to 200, 200 to 400, 400 to 600, 600 to 800, 800 to 1,000, 1,000 to 1,200, 1,200 to 1,400, 1,400 to 1,600, 1,600 to 1,800, 1,800 to 2,000, 2,000 to 2,200, 2,200 to 2,400, 2,400 to 2,600, 2,600 to 2,800, 2,800 to 3,000, 3,000 to 3,200, 3,200 to 3,400, 3,400 to 3,600, 3,600 to 3,800, 3,800 to 4,000, 4,000 to 4,200, 4,200 to 4,400, 4,400 to 4,600, 4,600 to 4,800, or 4,800 to 5,000 mg per day.

In some examples, the therapeutically effective amount of the curcumin or a pharmaceutically acceptable salt thereof can be from 1 to 200, 200 to 400, 400 to 600, 600 to 800, 800 to 1,000, 1000 to 1200, or 1200 to 1400 mg/kg. In certain examples, the therapeutically effective amount of the curcumin or a pharmaceutically acceptable salt thereof can be from 1 to 100, 100 to 200, 200 to 300, 300 to 400, 400 to 500, 500 to 600, 600 to 700, 700 to 800, 800 to 900, 900 to 1,000, 1000 to 1200, or 1200 to 1400 mg/kg. Further, the therapeutically effective amount of the curcumin or a pharmaceutically acceptable salt thereof can be from 1 to 10000, 200 to 8000, 500 to 6000, 1000 to 4000, 2000 to 3000, or about 1200 mg/kg.

In some examples, the therapeutically effective amount of the curcumin or a pharmaceutically acceptable salt thereof can be from 1 to 1,000, 1,000 to 2,000, 2,000 to 3,000, 3,000 to 4,000, 4,000 to 5,000, 5000 to 6000, 6000 to 7000, or 8000 mg/kg per day. In certain examples, the therapeutically effective amount of the curcumin or a pharmaceutically acceptable salt thereof can be from 1 to 1,000, 1,000 to 2,000, 2,000 to 3,000, 3,000 to 4,000, 4,000 to 5,000, 5000 to 6000, 6000 to 7000, or 8000 mg/kg per day. Further, the therapeutically effective amount can be from 1 to 1,000, 1,000 to 2,000, 2,000 to 3,000, 3,000 to 4,000, 4,000 to 5,000, 5000 to 6000, 6000 to 7000, or 8000 mg/kg per day.

Pharmaceutical compositions disclosed in the invention may be prepared, packaged, or sold in formulations suitable for oral administration. The formulations of the pharmaceutical compositions described herein may be prepared by any method known or hereafter developed. In general, preparation includes bringing the active ingredient into association with a carrier or one or more other additional components, and then, if necessary or desirable, shaping or packaging the product into a desired single- or multi-dose unit.

As used herein, “additional components” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; demulcents; buffers; salts; thickening agents; fillers; emulsifying agents; antioxidants; stabilizing agents; pharmaceutically acceptable polymeric or hydrophobic materials, as well as other components and agents.

A tablet comprising the drug may be made, for example, by compressing or molding the drug, optionally containing one or more additional components. Compressed tablets may be prepared by compressing, in a suitable device, the drug in a free-flowing form such as a powder or granular preparation, and then optionally mixing with one or more of a binder, a lubricant, a glidant, an excipient, a surface active agent and a dispersing agent. Molded tablets may be made by molding in a suitable device, a mixture of the drug, a pharmaceutically acceptable carrier, and at least sufficient liquid to moisten the mixtures.

Tablets may further comprise a sweetening agent, a flavoring agent, a coloring agent, a preservative, or some combination of these in order to provide pharmaceutically elegant and palatable preparations.

Hard capsules comprising the pharmaceutical agent may be made using a physiologically degradable composition, such as gelatin. Such hard capsules comprise the active ingredient, and may further comprise additional components including, for example, an inert solid diluent. Soft gelatin capsules comprising the pharmaceutical agent may also be made using a physiologically degradable composition, such as gelatin. Such soft capsules comprise the pharmaceutical agent, which may be mixed with water or an oil medium.

Tablets and pills of the present invention can additionally be prepared with release-controlling coatings. Such a coating may be colored with a pharmaceutically accepted dye. The amount of dye and other excipients in the coating may vary. The coating generally comprises film-forming polymers such as hydroxy-propyl cellulose, hydroxypropylmethyl cellulose, cellulose ester, or ether, in acrylic polymer or a mixture of polymers. The coating solution is generally an aqueous solution that may further comprise propylene glycol, sorbitan monooleate, sorbic acid, or fillers such as titanium dioxide, a pharmaceutically acceptable dye.

The solid pharmaceutical compositions of the present invention may further include diluents. Diluents for solid compositions include, for example, microcrystalline cellulose (e.g. AVICEL®), silicified microcrystalline cellulose, microfine cellulose, lactose, starch, pregelatinized starch, calcium sulfate, sugar, dextrates, dextrin, dextrose, dibasic calcium phosphate dihydrate, tribasic calcium phosphate, kaolin, magnesium oxide, maltodextrin, mannitol, dextrates (e. g. EMDEX), hydrated dextrates, polymethacrylates (e.g. Eudragit®), potassium chloride, powdered cellulose, sodium chloride, sorbitol and talc.

Solid pharmaceutical compositions of the present invention may further include binders, e.g., acacia, alginic acid, carbomer (e.g., carbopol), carboxymethylcellulose sodium, dextrin, ethyl cellulose, gelatin, guar gum, hydrogenated vegetable oil, hydroxyethyl cellulose, hydroxypropyl cellulose (e.g., KLUCEL®), hydroxypropyl methyl cellulose (e.g. METHOCEL®), liquid glucose, magnesium aluminum silicate, maltodextrin, methylcellulose, polymethacrylates, povidone (e.g., KOLLIDON®, PLASDONE®), pregelatinized starch, sodium alginate and starch.

Solid pharmaceutical compositions of the present invention may further include disintegrants such as alginic acid, carboxymethylcellulose calcium, carboxymethylcellulose sodium (e.g., AC-DI-SOL®, PRIMELLOSE®), colloidal silicon dioxide, croscarmellose sodium, crospovidone (e.g., KOLLIDON®, POLYPLASDONE®), guar gum, magnesium aluminum silicate, methyl cellulose, polacrilin potassium, powdered cellulose, pregelatinized starch, sodium alginate, sodium starch glycolate (e.g., EXPLOTAB®), hydroxypropylcellulose, methylcellulose, povidone or starch. Glidants, such as, colloidal silicon dioxide, magnesium trisilicate, powdered cellulose, starch, talc, and tribasic calcium phosphate may also be added.

Other pharmaceutical additives of the present invention may include: (i) lubricants such as magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate; (ii) flavoring agents and flavor enhancers such as vanillin, ethyl vanillin, menthol, citric acid, fumaric acid ethyl maltitol, and tartaric acid; (iii) pharmaceutically acceptable colorants; (iv) artificial sweeteners such as polyhydric alcohols, e.g., sorbitol, mannitol, xylitol, saccharin, saccharin sodium, aspartame, sucralose and maltitol; and, (v) natural sweeteners, such as glucose, fructose, sucrose and the like.

By way of non-limiting illustration, examples of certain embodiments of the present disclosure are given below.

### Examples

Reference will now be made to more specific embodiments of the present disclosure and experimental results that provide support for such embodiments. However, Applicants note that the disclosure below is for illustrative purposes only and is not intended to limit the scope of the claimed subject matter in any way.

Ursolic acid inhibited initiation, progression, and metastasis of prostate cancer in TRAMP mice by modulating pro-inflammatory signaling pathways and promotes prostate cancer apoptosis. There are no current prostate cancer clinical trials of ursolic acid. The estimated human equivalent dose for ursolic acid is 22 mg/kg per day. Many men presenting to clinic are at least 70 kg; thus, a dose of >1.5 grams per day would be needed or around 750 mg with twice a day dosing. Several trials are testing oral administration of ursolic acid for diabetes (150 mg daily after fasting, NCT02337933) and to improve muscle function (500 mg/day, NCT02401113). We initiate our dose at 300 mg daily.

Curcumin has been studied in several clinical trials without side effects to doses up to 8,000 mg/day. A current prostate clinical trial has been reported in Clinicaltrials.gov testing 1000 mg/day after prostatectomy to prevent cancer recurrence (NCT02064673), and another study with curcumin (3,000 mg/day) as an adjuvant to radiation therapy (NCT01917890). Based on our lipid-based formulation, we tested a dose of 1200 mg/day.

Curcumin and Ursolic Acid Capsule Manufacturing: The capsules were designed with Southwest Research Institute (SwRI) using good manufacturing practice (GMP) techniques. After purchasing ursolic acid and curcumin, we performed milling to reduce particle size and added lipid excipients to formulate a microsphere. Our final product was loaded into cellulose capsules.

Study Design: We performed a standard 3 by 3 phase 1 design based on the safety evaluation of curcumin and ursolic acid tested individually, then in combination (Study Schema in FIG. 4). We modified the 3 by 3 design to include any toxicities because we selected the maximum dose due to the storied safety with large doses of these compounds.

Population and Recruitment: We recruited healthy men enrolled in the SABOR study, which is a community-based prostate cancer screening cohort. The San Antonio Biomarkers of Risk (SABOR) study cohort was funded by the National Cancer Institute Early Detection Research Network-sponsored Clinical and Epidemiologic Validation Center since its nascent enrollment in 2000 (IRB #HSC20000030H). A separate IRB was approved for the phase 1 trial (IRB #HSC20190940H). Subjects had no prior diagnosis of prostate cancer at the time of enrollment and varied prostate cancer risk profiles. We used the local UT Health San Antonio Data Safety and Monitoring Board (DSMB).

Study Outcomes: Safety was compared to evaluate the number, frequency, duration, and relation of toxicity events to CURC and UA, as defined by the Common Terminology Criteria for Adverse Events (CTCAE) v 4.03. Secondary outcomes included the measured plasma level of ursolic acid, curcumin, and their metabolites. We also investigated the impact on the microbiome in a paired analysis for change and overall theme of imputed bacterial function.

Measurement of Compounds: The compounds and their metabolites were measured using an UHPLC-MS/MS system performed on a Hybrid quadrupole-Orbitrap mass spectrometer (Q Exactive, Thermo Scientific, Waltham, MA, USA) hyphenated with a Thermo Scientific Vanquish Flex ultrahigh-pressure liquid chromatography (UHPLC) system via electrospray ionization source. For the parallel reaction monitoring (PRM) scan, the resolution, auto gain control (AGC) target, and maximum injection time (IT) were set at 17500, 2e5, and 100 ms. The NCE value for each metabolite was individually set, and the details are provided in the table below. The All-Ion Fragmentation (AIF) scan consisted of a scan range of m/z 100-750 with NCE 10. The resolution, AGC target, and maximum IT for the AIF scan were set at 70000, 3e6, and 200 ms, respectively.

| | | | Major | | | | Linear | Linear | | Analyte | IS Used |
|---|---|---|---|---|---|---|---|---|---|---|---|
| | RT | | Transition/ | | | LLOQ | Range | Regression | Calibration | used for | for |
| Analytes/IS | (min) | [M − H]− | Base Peak | NCE | Mode | (ng/mL) | (ng/mL) | Equation & R2 | Type | Calibration | Calibration |
| Curcumin | 8.40 | 367.38 | 149.06 | 20 | PRM | 0.1 | 0.1-1000 | Y = | Internal | Curcumin | Curcumin-d₆ |
| | | | | | | | | 0.00563874 + | Calibration | | |
| | | | | | | | | 0.00193131*X | | | |
| | | | | | | | | R\hat{ }2 = 0.9983 | | | |
| Curcumin | 7.16 | 447.44 | 217.07 | 20 | PRM | N/A | N/A | N/A | External | Curcumin | Curcumin-d₆ |
| Sulfate | | | | | | | | | Calibration | Sulfate-d₆ | |
| Curcumin β-D- | 6.61 | 543.50 | 113.02 | 15 | PRM | N/A | N/A | N/A | External | Curcumin β-D- | Curcumin-d₆ |
| Glucuronide | | | | | | | | | Calibration | Glucuronide-d₃ | |
| Curcumin | 7.16 | 453.00 | 220.07 | 20 | PRM | 1 | 1-1000 | Y = | External STD | N/A | N/A |
| Sulfate-d₆ | | | | | | | | 0.000542484 + | to quantify | | |
| | | | | | | | | 0.000370986*X | Curcumin | | |
| | | | | | | | | R\hat{ }2 = 0.9968 | Sulfate | | |
| Curcumin β-D- | 6.61 | 546.50 | 113.02 | 15 | PRM | 0.5 | 0.5-1000 | Y = | External STD | N/A | N/A |
| Glucuronide-d₃ | | | | | | | | 0.000943129 + | to quantify | | |
| | | | | | | | | 0.000203698*X | Curcumin | | |
| | | | | | | | | R\hat{ }2 = 0.9973 | Glucuronide | | |
| Curcumin-d₆ | 8.40 | 373.42 | 152.08 | 20 | PRM | N/A | N/A | N/A | IS | N/A | N/A |
| Betulinic | 7.63 | 455.70 | 455.25 | 10 | AIF | N/A | N/A | N/A | IS | N/A | N/A |
| acid | | | | | | | | | | | |
| Ursolic acid | 11.77 | 455.70 | 455.35 | 10 | AIF | 1 | 1-1000 | Y = | Internal | Ursolic acid | Betulinic |
| | | | | | | | | 0.0235383 + | Calibration | | acid |
| | | | | | | | | 0.00196138*X | | | |
| | | | | | | | | R\hat{ }2 = 0.9978 | | | |

### CYP3A IC50 Determination:

Microbiome Methods: We obtained a rectal swab from participants at baseline visit and after 2 weeks and isolated DNA using our previously published methods. Genomic DNA was used for amplification of V1-V2 variable region of the 16S rRNA genes with custom-designed primers (F27/R534, and V3-V4 variable region of the 16S rRNA genes following the Illumina 16S metagenomic library preparation guide. Final libraries were quantified, normalized, pooled together, and sequenced by Paired-end sequencing (2× 300 bp) using Illumina Miseq platform. Raw paired-end 16S rRNA read sets were merged into consensus fragments and confirmed. Primary taxonomic assignment was performed using closed-reference OTU picking the against GreenGenes database using default parameters. PICRUSt was applied to rarefied taxonomic profiles to infer functional categories associated with taxonomic composition. Alpha and beta-diversity analyses were performed using QIIME. Primary differential abundance analysis between baseline and 2-weeks per treatment employed the paired T-test with log-transformation of feature values, supplemented by the paired Mann-Whitney test and unpaired significance tests for group level comparisons. All computational analyses were performed on V1V2 and V3V4 amplicon regions separately.

Statistical Analysis: Demographics between groups was compared using the Fischer Exact test for categorical variables and the Student's t-test for continuous variables. Safety laboratory values were analyzed using a paired t-test for differences, with significance set at p<0.05. ANOVA was used to compare plasma levels of UA, CURC, and CURC metabolites. Least significant difference (LSD) was used as the post hoc analysis test to compare values between groups.

### Results

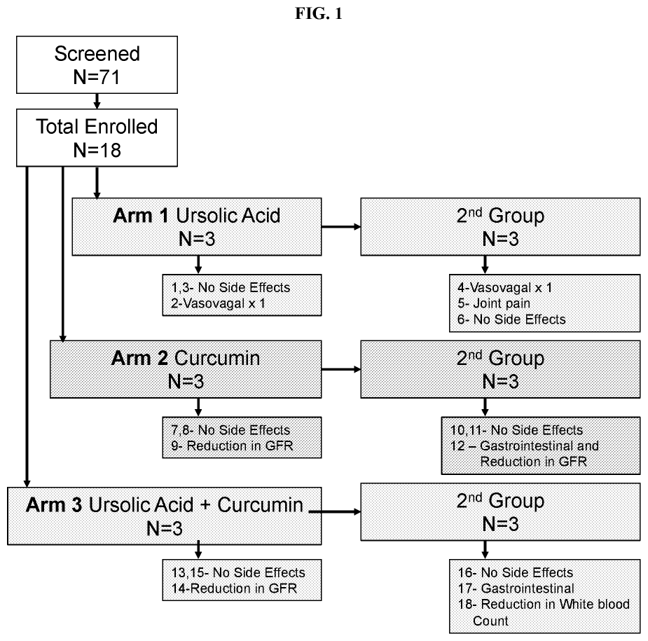

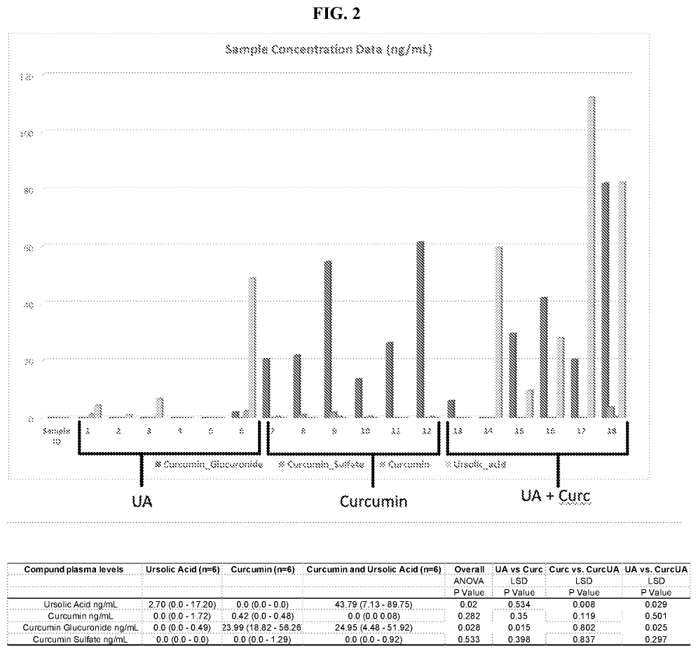

Population: We contacted 71 subjects based on active status and previous indications that they would be interested in subsequent studies (Consort Diagram, FIG. 1). We enrolled 18 subjects into a sequential trial design. Demographics are displayed in Table 1. There were no statistical differences in demographics between groups. All 18 subjects completed the study with zero drop-outs due to side effects.

TABLE 1
Demographics. The initial papers leading to the experiments were based on the future treatment of prostate cancer; therefore,
we enrolled healthy men to a phase 1 clinical trial with three cohorts that include ursolic acid (UA), curcumin (Curc) and
the combination (CurcUA). In the first column we show the overall median and interquartile range (IQR) of demographics including
some that are important during screening for prostate cancer. Then each of the 3 cohorts is displayed separately. The ANOVA
test (continuous values) and the Fischer's Exact (categorical values) is used to compare the group for differences.

| | | | | | | | Curcumin | | |
|---|---|---|---|---|---|---|---|---|---|
| | Total Cohort | | Ursolic Acid | | Curcumin | | and Ursolic | | |
| | (n = 18) | | (n = 6) | | (n = 6) | | Acid (n = 6) | | ANOVA |
| | Median (IQR) | | Median (IQR) | | Median(IQR) | | Median (IQR) | | P |
| Baseline Demographics | or Number (%) | | or Number (%) | | or Number (%) | | or Number (%) | | Value |
| Age | 67 | (60-72) | 69 | (59-75) | 70 | (64-74) | 62 | (60-67) | 0.24 |
| Race/Ethnicity | | | | | | | | | 0.41 |
| European / White | 12 | (67%) | 4 | (67%) | 4 | (67%) | 4 | (67%) | |
| Hispanic / Latino | 3 | (17%) | 2 | (33%) | 0 | (0%) | 1 | (17%) | |
| Black/African American | 3 | (17%) | 0 | (0%) | 2 | (33%) | 1 | (17%) | |
| Body Mass Index (BMI) | 25.6 | (24.2-31) | 24.7 | (24.0-27.4) | 25.0 | (23.1-32.8) | 30.1 | (25.2-31.5) | 0.523 |
| Active Smoker | 2 | (28%) | 3 | (50%) | 0 | (0%) | 2 | (33%) | 0.14 |
| Diabetes | 3 | (17%) | 2 | (33%) | 1 | (17%) | 0 | (0%) | 0.47 |
| Hpercholesterolemia | 4 | (22%) | 2 | (33%) | 2 | (33%) | 0 | (0%) | 0.28 |
| Hypertention | 5 | (28%) | 3 | (50%) | 2 | (33%) | 0 | (0%) | 0.14 |
| Family history of prostate cancer | 5 | (28%) | 2 | (33%) | 2 | (33%) | 1 | (17%) | 0.76 |
| Prostate Specific Antigen (PSA) | 0.95 | (0.73-1.38) | 1.00 | (0.38-2.23) | 0.95 | (0.73-1.48) | 1.1 | (0.68-1.38) | 0.72 |
| Abnormal prostate exam | 0 | (0%) | 0 | (0%) | 0 | (0%) | 0 | (0%) | 0.99 |
| Previous Negative Prostate Biopsy | 2 | (11%) | 2 | (33%) | 0 | (0%) | 0 | (0%) | 0.11 |
| Prostate Size on Exam | | | | | | | | | |
| Smal (<30 gm) | 12 | (67%) | 4 | (67%) | 4 | (67%) | 4 | (67%) | 0.99 |
| Medium (30-60 gm) | 6 | (33%) | 2 | (33%) | 2 | (33%) | 2 | (33%) | 0.99 |
| Large (>60 gm) | 0 | (0%) | 0 | (0%) | 0 | (0%) | 0 | (0%) | 0.99 |
| Medications for Bengin | 0 | (0%) | 0 | (0%) | 1 | (17%) | 1 | (17%) | 0.57 |
| prostate hypertrophy (BPH) | | | | | | | | | |
| Previous Antibiotics | 2 | (11%) | 2 | (33%) | 0 | (0%) | 0 | (0%) | 0.11 |
| in the last 6 months | | | | | | | | | |
| Previous vitamin or | 14 | (78%) | 4 | (67%) | 5 | (83%) | 5 | (83%) | 0.73 |
| nutritional supplment use | | | | | | | | | |

Confirmation of capsule ingredients: The contents of the raw materials were found to be 91.0% Ursolic acid and 95.7% curcumin. Further analyses were performed on the capsules after milling, lipid microspheres preparation, and addition of excipients. No alterations to the parent compounds were noted as 100% curcuminoids and 100% ursolic acid structures were noted. The single capsule content was 41% curcumin (200 mg) and 12.6% UA (50 mg). Both capsules exhibited no microbial contamination.

Safety: Overall the supplements were well tolerated, and no one stopped the trial due to side effects.

Common Terminology Criteria for Adverse Events (CTCAE) v 4.03. We have summarized the CTCAE forms in Table 2. A common low-grade subject complaint was gastrointestinal (GI) related and more attributed to ursolic acid (n=3). There were two neurological domain incidents. One vasovagal reaction that occurred with the initial blood draw prior to drug ingestion and a presyncope episode in a man that donated blood earlier in the day both in the curcumin arm. We noted a rise in creatinine in three men all who took ursolic acid, two in the UA alone and one in the combination group. A creatinine rise would indicate kidney dysfunction. These were laboratory values only and no long term effects or treatment was needed (Grade 1).

TABLE 2
Safety evaluation. Subjects were evaluated at each study visit for adverse events and side effects using the standardized Common Terminology
Criteria for Adverse Events (CTCAE) version 4.03. We have indicated the ID number, study group, if an event took place, the study grade,
domain, and attribution. Only one grade 3 even took place and was attributed to blood draw and not attributed to the study drug.

| | | | | Toxicity | | | | |
|---|---|---|---|---|---|---|---|---|
| | | | | Grade | | | Relationship | |
| ID | | Adverse | | (CTCAE | | | To Study | |
| Number | Group | Event | Event | v4.0) | Expected | Domain | Treatment | Comments |
| CU-CD-01 | Curcumin | No | | | | | | |
| CU-CD-02 | Curcumin | Yes | Vasovagal | 3 | N | Nervous system | Unlikely | Occurred prior to subjects first |
| | | | Reaction | | | | | dose. |
| CU-CD-03 | Curcumin | No | | | | | | |
| CU-CD-04 | Curcumin | Yes | Pre-synscope | 2 | N | Nervous system | Unlikely | Donated plasma earlier in the day. |
| CU-CD-05 | Curcumin | Yes | Joint pain | 1 | N | Musculoskeletal | Unlikely | Mild joint pain. |
| CU-CD-06 | Curcumin | No | | | | | | |
| CU-CD-07 | Ursolic Acid | No | | | | | | |
| CU-CD-08 | Ursolic Acid | No | | | | | | |
| CU-CD-09 | Ursolic Acid | Yes | Creatinine | 1 | N | Blood/Urinary | Unlikely | Creatine increased from 1.10 to |
| | | | increased | | | | | 1.24. |
| CU-CD-10 | Ursolic Acid | No | | | | | | |
| CU-CD-11 | Ursolic Acid | No | | | | | | |
| CU-CD-12 | Ursolic Acid | Yes | Diarrhea | 1 | Y | GI | Possible | |
| CU-CD-12 | Ursolic Acid | Yes | Bloating | 2 | Y | GI | Possible | |
| CU-CD-12 | Ursolic Acid | Yes | Flatulence | 1 | Y | GI | Possible | |
| CU-CD-12 | Ursolic Acid | Yes | Creatine | 1 | N | Blood/Urinary | Unlikely | Creatine increased from 1.0 to |
| | | | increased | | | | | 1.21. |
| CU-CD-13 | Curc + UA | No | | | | | | |
| CU-CD-14 | Curc + UA | Yes | Creatinine | 1 | N | | Unlikely | Creatine increased from 1.13 to |
| | | | increased | | | | | 1.32. |
| CU-CD-15 | Curc + UA | No | | | | | | |
| CU-CD-16 | Curc + UA | No | | | | | | |
| CU-CD-17 | Curc + UA | Yes | Flatulence | 1 | Y | GI | Possible | |
| CU-CD-18 | Curc + UA | Yes | White blood | 2 | N | Blood/Urinary | Unlikely | Baseline low WBC count (3.1 to |
| | | | cell decreased | | | | | 2.4). |

Laboratory Safety values. We perform laboratory analysis at baseline and at the end of the two-week study. We then subtracted the values (week 2-baseline) to obtain the differences for each participant. The summary values are displayed in Table 3 showing the median difference and IQR between values.

TABLE 3
Safety laboratory results. We display the differences between the week 2 values minus the baseline values. A minus sign
will show a reduction in that value over 2 weeks. The p values were calculated using a paired T test for difference, where
P < 0.05 is a significant difference. While there was a significant reduction in potassium in the curcumin cohort
this was not clinically significant. In the CurcUA group, the statistically significant differences in bicarbonate, creatinine,
glomerular filtration rate (GFR), and alanine aminotransferase (ALT) were not clinically significant changes.

| Difference in Saftey | | | Ursolic Acid + | UA | Curc | CurcUA |
|---|---|---|---|---|---|---|
| Laboratory Tests | Ursolic Acid (n = 6) | Curcumin (n = 6) | Curcumin | (P value) | (P value) | (P value) |
| White blood cell (WBC) | 0 20 (−0.23 tom 0.85) | 0.15 (−0.43 to 0.68) | 0.15 (−0.73 to 0.38) | 0.25 | 0.52 | 0.53 |
| Hemoglobin | 0.20 (−0.33 to 0.58) | 0.20 (−0.30 to 0.55) | 0.05 (−0.55 to 0.42) | 0.66 | 0.37 | 0.76 |
| Hematocrit | 1.55 (−0.85 to 2.85) | 0.25 (−1.45 to 2.35) | 0.35 (−2.18 to 1.53) | 0.39 | 0.74 | 0.71 |
| Platelets | 9.5 (2.5 to 27) | 7.0 (−5 to 31.5) | 0.0 (−7.5 to 8.0) | 0.08 | 0.28 | 0.956 |
| Sodium | 0.5 (−1.5 to 2.25) | 0.0 (−1.5 to 1.0) | 0.05 (−0.8 to 1.3) | 0.72 | 0.61 | 0.82 |
| Potassium | 0.15 (−0.08 to 0.45) | −0.15 (−0.3 to −0.8) | −0.05 (−0.3 to 0.2) | 0.1 | 0.02 | 0.9 |
| Chloride | −1.0 (−2.75 to 1.75) | −0.5 (−1.3 to 0.8) | 0.5 (−1.5 to 2.0) | 0.85 | 0.82 | 0.84 |
| Bicarbonate | 1.0 (−1.5 to 3.0) | −1.5 (−4.3 to 0.0) | −2.5 (−3.3 to −0.5) | 0.48 | 0.21 | 0.04 |
| Creatinine | −0.08 (−0.33 to 0.12) | 0.07 (−0 07 to 0.16) | 0.06 (0.03 to 0.98) | 0.18 | 0.38 | 0.04 |
| Glomerular Filtration | −3.5 (−10.3 to 3.5) | −5.5 (−12.0 to 4.5) | −4 (−7.3 to −1.8) | 0.36 | 0.54 | 0.03 |
| Rate (GFR) | | | | | | |
| Calcium | 0.03 (−0.05 to 0.48) | 0.2 (−0.15 to 0.32) | 0.1 (−0.23 to 0.32) | 0.14 | 0.6 | 0.59 |
| Bilirubin | 0.0 (−0.08 to 0.15) | −0.1 (−0.3 to 0.0) | 0.05 (−0.13 to 0.23) | 0.84 | 0.06 | 0.56 |
| Albumin | 0.0 (−0.1 to 0.2) | 0.05 (−0.05 to 0.2) | 0.0 (−0.13 to 0.2) | 0.61 | 0.46 | 0.81 |
| AST | 2.0 (−1.3 to 3.3) | 2.0 (−2.0 to 3.3) | −1.0 (−2.3 to 0.8) | 0.25 | 0.08 | 0.81 |
| ALT | 1.5 (1.0 to 12.8) | −1.0 (−3.25 to 0.0) | −3.5 (−5.0 to −1.5) | 0.16 | 0.54 | 0.01 |
| Alkaline Phosphatase | 2.5 (−2.5 to 5.0) | −2.0 (−5.5 to 9.3) | 0.5 (−5.5 to 9.5) | 0.35 | 0.81 | 0.56 |

Renal function: On review of the lab values, we noted a concern regarding kidney function. Chronic kidney (CKD) disease is based on GFR, and CKD stage 3 starts at a value below 60. One percipient enrolled in the curcumin arm with a baseline GFR of 45 and after 2 weeks, value was 46. Only one participate started >60 GFR and dropped below that number. The participant also had the largest drop in GFR (17 points) from 76 to 59 in the combination (CurcUA) group. However, his plasma values of both curcumin and ursolic acid were undetectable and only had a small amount of curcumin glucuronide. Therefore, it would be difficult to attribute poor kidney function to high ursolic acid levels. Despite this, the creatinine rises and subsequent lowering of GFR did not result in clinically meaningful side effects. However, in future studies kidney function should be closely monitored.

Bicarbonate. Bicarbonate can be harmful if large changes happen in either direction but usually clinicians are concerned about metabolic acidosis in most cases. We noted bicarbonate to be statistically significantly different between baseline and two weeks in the combination group (CurcUA). In the case of the bicarbonate value in the study they reduced the value by 2.5 to be less acidic. The situation may be beneficial to normal patients yet the value of 2.5 difference was of inconsequential clinical significance.

Liver function. No patients had outward signs of liver complications such as jaundice or pruritis. The alanine aminotransferase (ALT) was significantly difference than baseline. Concerning liver functions will rise with time; however, we noted a reduction in ALT by 3.5 in the combination arm (CurcUA). The value would suggest improvement rather than harm yet is still of inconsequential clinical significance.

Peak Plasma Levels (Cₘₐₓ): The maximum concentration at day 14 was compared across groups. In the UA alone cohort (Group 1: UA), UA plasma values reached a median of 2.7 ng/ml (IQR 0.0-17.2 ng/ml) and were not statistically different than in the CURC alone group that did not ingest UA (p=0.5). The median plasma values of CURC and its metabolites in this group were 0.0 ng/mL as expected. In the group assigned to CURC alone (Group 2: CURC), the median plasma level of the parent compound was only 0.42 ng/ml (IQR 0.0-0.48 ng/mL). The highest level of CURC was 0.52 ng/ml. The major metabolite detected was CURC glucuronide with a median level of 24.0 ng/ml (IQR 18.8-56.3 ng/ml). There were no significant differences in CURC (0.42 vs. 0.0 ng/ml, p=0.1) or CURC glucuronide (24 ng/mL vs. 25 ng/mL, P=0.8) plasma levels between the CURC alone group and the combination (CurcUA) groups, respectively. UA was not detected in the CURC only treated group as expected. In the combination group (Group 3: CurcUA), the CURC levels were low and similar to that observed in both the UA and CURC only groups. Notably, the plasma levels of UA increased by 16-fold (from a median of 2.7 ng/ml to 43.8 ng/mL) in the combination treatment group (P=0.03).

CYP3A4: To explore potential mechanisms for the altered plasma levels of UA when given in combination with CURC, we determined IC50 values.

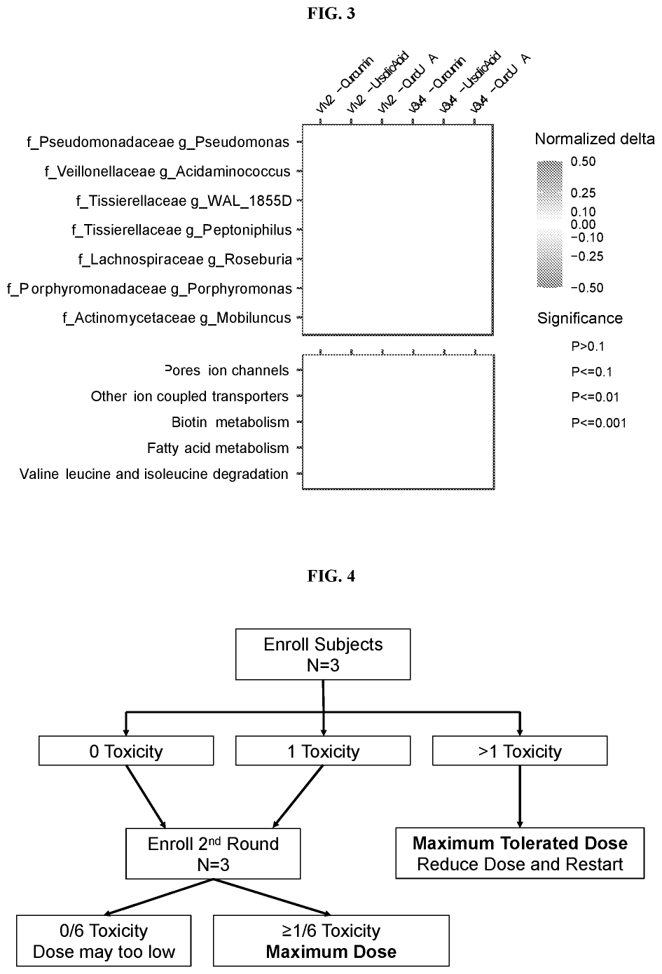

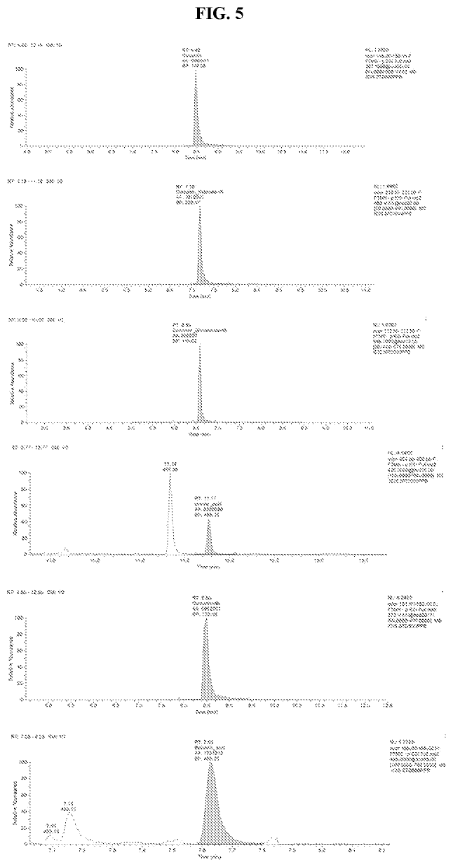

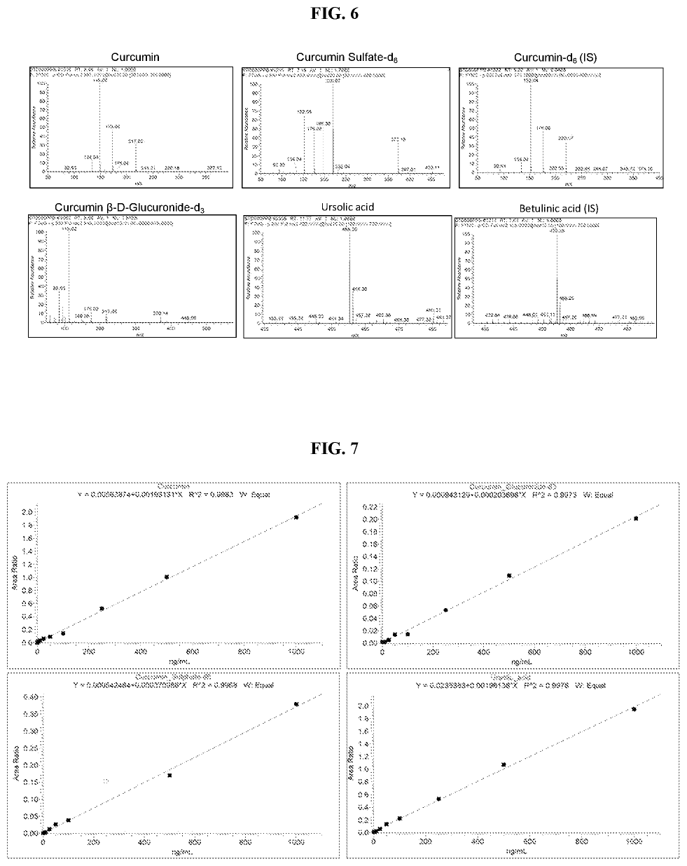

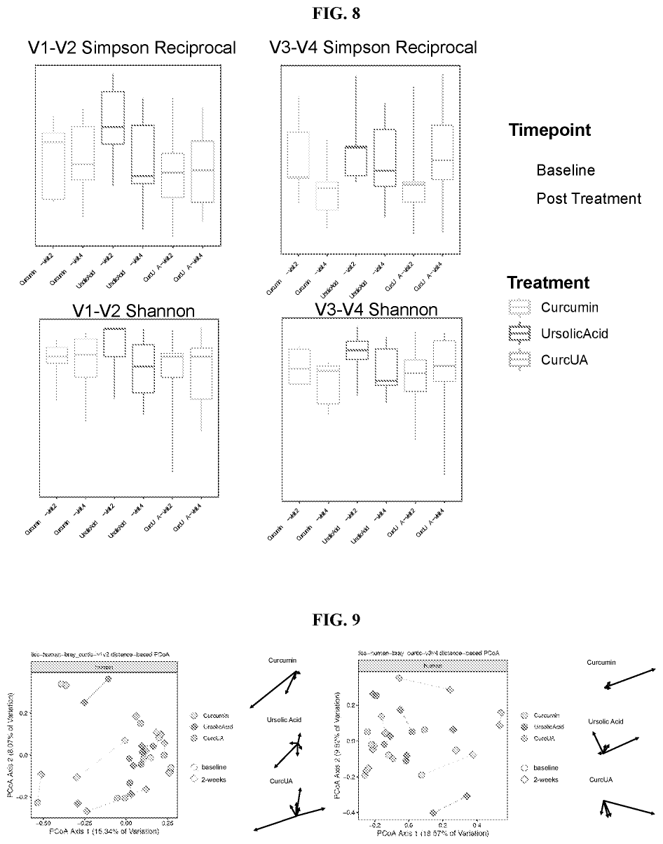

Microbiome: Both V1-V2 and V3-V4 techniques were used to explore if alpha diversity as a measure of richness and evenness of a gut bacteria changed over the 2-week study period. Thus, the pre and post diversity values were compared using 10 different diversity tests but displayed the Simpson's Reciprocal Index and the Shannon index in box plots in FIG. 8. The only significant value was a reduction in alpha diversity in the V3-V4 Shannon index for CURC (p=0.004). There was a trend (p=0.06) in the combination group (CurcUA) toward increased bacterial diversity in V3-V4 Simpson reciprocal index. The beta diversity as a measure of similarity between groups noted significant differences using 6 difference analysis technique; all values were significant (p<0.05, Table 4). We display a principal coordinate plot (PCoA) in FIG. 9 to show that the diversity was more associated with the individual person than the group of treatment assigned. Individually the treatments changed the gut microbiome, but we did not see significant grouping over the short time. In FIG. 3, we provide a summary of analysis to determine specific bacteria and metabolic pathways impacted by the groups. Bacterial associations included changes in dysbiotic bacteria (Pseudomonas, Acidaminococcus, Tissierellaceae, and Mobiluncus) and healthy bacteria (Roseburia and Porphyromonas). Of the most interesting of the metabolic pathways, biotin metabolism has been associated with a reduced prostate cancer risk. Fatty acid metabolism has been a target for prostate cancer prevention and treatment.

TABLE 4
The table shows the various statistical comparisons used to compare the beta diversity noting a statistically
significant difference using several different computational comparisons. After adjusting for patient-to-patient
variation and timepoint, gut microbiota composition is associated with at least 1 treatment exposure.

| | PERMANOVA evaluation of Tx Exposure Effect | | | | | | |
|---|---|---|---|---|---|---|---|
| Algorithm-distance-region | Df | SumsOfSqs | MeanSqs | F. Model | R2 | Pr (>F) | |
| liss-human-bray_curtis-v1v2 | 2 | 0.999 | 0.4995 | 4.6224 | 0.08965 | 0.0002 | *** |
| liss-human-bray_curtis-v3v4 | 2 | 0.7356 | 0.36782 | 3.5862 | 0.08354 | 0.0002 | *** |
| liss-human-unweighted_unifrac-v1v2 | 2 | 0.4292 | 0.21462 | 3.0867 | 0.0858 | 0.0002 | *** |
| liss-human-unweighted_unifrac-v3v4 | 2 | 0.3882 | 0.19412 | 2.3448 | 0.08745 | 0.0002 | *** |
| liss-human-weighted_unifrac-v1v2 | 2 | 1.4002 | 0.70008 | 3.7461 | 0.08983 | 0.0002 | *** |
| liss-human-weighted_unifrac-v3v4 | 2 | 0.13724 | 0.06862 | 2.6492 | 0.08981 | 0.02 | * |
| insight-human-bray_curtis-v1v2 | 2 | 0.7496 | 0.37482 | 3.4679 | 0.08144 | 0.0002 | *** |
| insight-human-bray_curtis-v3v4\| | 2 | 0.6383 | 0.31916 | 3.2705 | 0.0851 | 0.0002 | *** |
| insight-human-unweighted_unifrac-v1v2 | 2 | 0.5267 | 0.26336 | 2.1036 | 0.07778 | 0.0002 | *** |
| insight-human-unweighted_unifrac-v3v4 | 2 | 0.6562 | 0.32812 | 1.6093 | 0.08196 | 0.0002 | *** |
| insight-human-weighted_unifrac-v1v2 | 2 | 2.149 | 1.07449 | 3.6299 | 0.0927 | 0.0002 | *** |
| insight-human-weighted_unifrac-v3v4 | 2 | 0.12739 | 0.06369 | 2.3751 | 0.07198 | 0.0114 | * |

### Discussion

Using a phase 1 clinical trial, a 16-fold increase in plasma levels of orally dosed UA was observed when given in a combination with CURC. CURC (parent compound) levels were low in plasma regardless of whether it was given alone or in combination of UA. CURC in plasma was found primarily in its glucuronidated form as CURC glucuronide, which has been shown to be less biologically active than the parent compound. Thus, the presence of CURC given together with UA increased the overall bioavailability of UA.

CURC metabolism is driven by glucuronidation that may occur from bacterial interactions (E. coli), intestinal cells, or liver cells. Overall changes in the abundance of fecal E. coli abundance between groups was not observed in the current study. One study indicated that nearly 60% of CURC is excreted as parent compound in the feces among other absorptions issues. In a previous phase 1 dose-escalation study, healthy subjects took one-time doses of curcumin ranging from 0.5 to 12 grams. The investigators noted that subjects would need to ingest more the 8 grams per day to attain a measurable level the parent compound curcumin in the blood. Another study on subjects with Alzheimer's disease (n=36) were tested at 2 grams or 4 grams per day for 24 weeks and had similar results to ours regarding side effect profile and blood levels. In that study, three subjects (8%) dropped out due to gastrointestinal side effects and overall had comparable curcumin (7 ng/ml) and curcumin glucuronide (96 ng/mL) plasma levels accounting for higher doses and longer duration. In the current study, the majority of CURC in the circulation was the glucuronide. In vivo studies have shown this form of CURC to be largely inactive against cancer cells.

While curcumin is rapidly metabolized, low levels of UA are largely due to poor intestinal absorption to leading to attempts at improving bioavailability in animal models. UA is a lipophilic pentacyclic triterpenoid, especially abundant in apple peel, holy basil, and cranberries and touted to have many benefits. The metabolism of UA includes a phase I metabolic route of olefin oxidation and phase II metabolic routes of glycine conjugation, glutathione conjugation and glucuronidation. The parent compound (UA) has been shown to prevent and reduce progression of prostate cancer in TRAMP mouse models. Other in vivo studies have shown that UA activates the peroxisome proliferator-activated receptor alpha (PPARα) linked to fatty acid uptake in high fat diets as an indirect method that could impact prostate cancers link with obesity.

Ursolic acid plus curcumin can also work indirectly through microbiome manipulation. UA is shown to increase beneficial bacteria, such as the phylum Firmicutes and the genera Lactobacillus and Bifidobacterium in mice to prevent liver fibrosis. Additionally, UA is an anti-inflammatory that protects against the effects of ulcerative colitis, as disease which has been linked to a fourfold increased risk of prostate cancer in a case-control evaluation of 10,000 men in the United States. A second population-based study performed using the UK-biobank noted a more modest but significant increase in risk in prostate cancer in those with UC (n=218,084, median follow-up 6.5 years, HR 1.47, 95% CI=1.11-1.95, P=0.007) and was confirmed in systematic reviews. No studies have yet elucidated the effects of UA in the human gut microbiome.

Curcumin also manipulates the microbiome and largely published as beneficial to the mouse microbiome and improves intestinal barrier function of the gut. One study investigating human microbiome and curcumin randomized 14 subjects and noted that curcumin increase the gut diversity by 67% compared to placebo that had a reduction in diversity by 15%. Curcumin has also been shown to reduce inflammation in mice and humans with ulcerative colitis.

Regarding the microbiome and prostate cancer, several studies have shown associations with the gut microbiome. The overall trend is that curcumin and ursolic acid seem to slightly reduce biodiversity of the gut microbiome, but the combination tends to improve overall diversity. The biodiversity of each individual was unique and did not cluster after treatment but did have changes in composition. Using two types of variable regions (V1-V2 and V3-V4) allowed us to visualize if there is consistency in the analysis. Those consistent across the variable regions are more robust. The only consistent changes were a reduction in Rosburia with curcumin, which is an unwanted side effect of curcumin as Rosburia is associated with a healthy gut microbiome.

Our group has published the metabolomic biosynthesis pathways related to prostate cancer diagnosis noting increases in starch metabolism was associated with cancer and bacterial biosynthesis of B vitamins (biotin and folate) associated with non-cancer. Biotin metabolism was significantly increased in the combination group across variable regions. A group in Japan investigated fecal microbiome associated with high-risk prostate cancer largely showing an increased abundance of bacteria that produce short chain fatty acids. Total short-chain fatty acids (SCFA) are linked to starch intake. For example, SCFA were significantly higher in the feces of animals fed the starch diet compared with those fed the sucrose diet.

1. A method of treating, inhibiting initiation, inhibiting progression, and/or inhibiting metastasis of cancer in a subject, comprising: administrating ursolic acid or a pharmaceutically acceptable salt thereof and curcumin or a pharmaceutically acceptable salt thereof.

2. The method of claim 1, wherein the cancer is prostate cancer.

3. The method of claim 1, wherein the ursolic acid or pharmaceutically acceptable salt thereof and the curcumin or pharmaceutically acceptable salt thereof are administered orally.

4. The method of claim 1, wherein the ursolic acid or pharmaceutically acceptable salt thereof and the curcumin or pharmaceutically acceptable salt thereof are administered together.

5. The method of claim 1, wherein the ursolic acid or pharmaceutically acceptable salt thereof and the curcumin or pharmaceutically acceptable salt thereof are together in a capsule when administered.

6. The method of claim 1, wherein the ursolic acid or pharmaceutically acceptable salt thereof is administered at from 150 mg/day to 500 mg/day.

7. The method of claim 1, wherein the ursolic acid or pharmaceutically acceptable salt thereof is administered at 300 mg/day.

8. The method of claim 1, wherein the curcumin of pharmaceutically acceptable salt thereof is administered at from 100 mg/day to 8,000 mg/day.

9. The method of claim 1, wherein the curcumin of pharmaceutically acceptable salt thereof is administered at 1200 mg/day.

10. A composition comprising ursolic acid or a pharmaceutically acceptable salt thereof and curcumin or a pharmaceutically acceptable salt thereof.

11. The composition of claim 10, wherein the composition comprises from 50 to 500 mg of ursolic acid or pharmaceutically acceptable salt thereof.

12. The composition of claim 10, wherein the composition comprises 300 mg of ursolic acid or pharmaceutically acceptable salt thereof.

13. The composition of claim 10, wherein the composition comprises from comprises from 50 to 8000 mg of curcumin or pharmaceutically acceptable salt thereof.

14. The composition of claim 10, wherein the composition comprises 1200 mg of curcumin or pharmaceutically acceptable salt thereof.

15. The composition of claim 10, wherein the composition further comprises a pharmaceutically acceptable excipient.
